(19) United States
(12) Patent Application Publication
(10) Pub. No.: US 2019/0133634 A1
(43) Pub. Date: May 9, 2019

(54) Micro-Needling System

(71) Applicants: Richard H. Ford, Braselton, GA (US); Eric Fuller, Cedartown, GA (US)

(72) Inventors: Richard H. Ford, Braselton, GA (US); Eric Fuller, Cedartown, GA (US)

(73) Assignee: Emvera Technologies, LLC, Cedartown, GA (US)

(21) Appl. No.: 16/181,709

(22) Filed: Nov. 6, 2018

Related U.S. Application Data
(60) Provisional application No. 62/581,821, filed on Nov. 6, 2017.

(51) Int. Cl. A61B 17/32; A61B 17/34

(52) U.S. Cl. CPC A61B 17/320068; A61B 2017/00761; A61B 17/3476

(57) ABSTRACT

A micro-needling device includes a hand-held motorized unit generates repetitive motion. An outer shell defines a cylindrical cavity therein and having a top surface. The top surface defines a plurality of needle holes passing therethrough. The outer shell is coupled to the hand-held motorized unit. A cylindrical plug member is disposed within the cylindrical cavity. A plurality of needles extends upwardly the plug member through the needle holes. A translation unit translates the repetitive motion from the hand-held motorized unit into oscillating inward and outward motion applied to the plug member so as to cause the plurality of needles to oscillate between an outward position in which the needles extend beyond the outer shell and an inward position as the rotary cam rotates about an axis.

### CROSS-REFERENCE TO RELATED APPLICATION(S)

This application claims the benefit of U.S. Provisional Patent Application Ser. No. 62/581,821, filed Nov. 6, 2017, the entirety of which is hereby incorporated herein by reference.

### BACKGROUND OF THE INVENTION

### 1. Field of the Invention

The present invention relates to skin treatment systems and, more specifically to a micro-needling skin treatment system.

### 2. Description of the Related Art

Several systems have been employed to rejuvenate skin. For example, personal micro dermabrasion (PMD) systems use a rotating disk to abrade the surface of the skin so as to reduce the appearance of fine lines and wrinkles, blemishes and enlarged pores. By removing the surficial dead skin cells, new cell growth is stimulated and circulation is increased, which replaces collagen in the skin.

Micro-needling is another skin treatment that involves using a tool small needles in the tip which are driven into the surface of the skin, usually to a depth of about 0.5 millimeters. Micro-needling creates micro-punctures from the needles the skin. In response thereto, the body sends fibroblasts to create more collagen in the affected area. The punctures can also facilitate absorption of emollients into the skin surface. However, the needles of existing micro-needling systems tend to push the skin surface away from the needles as the needles are being driven, which gives rise to uneven results.

Therefore, there is a need for a micro-needling system that applies needles to the skin in an even and consistent manner.

### SUMMARY OF THE INVENTION

The disadvantages of the prior art are overcome by the present invention which, in one aspect, is a micro-needling attachment for use with a hand-held motorized unit. An outer shell defines a cylindrical cavity therein and has a top surface. The top surface defines a plurality of needle holes passing therethrough. The outer shell is couplable to the hand-held motorized unit. A cylindrical plug member is disposed within the cylindrical cavity. A plurality of needles extends upwardly from the plug member through the needle holes. A translation unit translates motion of a first type from the hand-held motorized unit into oscillating inward and outward motion applied to the plug member so as to cause the plurality of needles to oscillate between an outward position in which the needles extend beyond the outer shell and an inward position.

In another aspect, the invention is a micro-needling attachment device for use with a hand-held motorized unit. An outer shell defines a cylindrical cavity therein and has a top surface. The top surface defines a plurality of needle holes passing therethrough. The outer shell is couplable to the hand-held motorized unit. A cylindrical plug member is disposed within the cylindrical cavity. The cylindrical plug member has a flat surface and an opposite uneven bottom surface that includes a base portion and a raised portion. A plurality of needles extends upwardly from the flat surface of the plug member through the needle holes. A rotatable lower rotary cam is disposed adjacent to the uneven bottom surface of the plug member. An axle depends downwardly therefrom and is engageable with and configured to receive rotational motion from the hand-held motorized unit, the rotary cam having an uneven cam surface that causes the plug member to oscillate between an outward position in which the needles extend beyond the outer shell and an inward position as the rotary cam rotates about an axis. A spring is disposed between the top surface of the outer shell and the flat surface of the cylindrical plug. The spring imparts a separating force between the top surface of the outer shell and the flat surface of the cylindrical plug so as to push the plug member into the inward position during a portion the rotary cam rotation cycle.

In yet another aspect, the invention is a micro-needling device. A hand-held motorized unit generates repetitive motion. An outer shell defines a cylindrical cavity therein and having a top surface. The top surface defines a plurality of needle holes passing therethrough. The outer shell is coupled to the hand-held motorized unit. A cylindrical plug member is disposed within the cylindrical cavity. A plurality of needles extends upwardly the plug member through the needle holes. A translation unit translates the repetitive motion from the hand-held motorized unit into oscillating inward and outward motion applied to the plug member so as to cause the plurality of needles to oscillate between an outward position in which the needles extend beyond the outer shell and an inward position as the rotary cam rotates about an axis.

These and other aspects of the invention will become apparent from the following description of the preferred embodiments taken in conjunction with the following drawings. As would be obvious to one skilled in the art, many variations and modifications of the invention may be effected without departing from the spirit and scope of the novel concepts of the disclosure.

### BRIEF DESCRIPTION OF THE FIGURES OF THE DRAWINGS

FIG. 1 is a top perspective view of one embodiment of a micro-needling system.

FIG. 2 is a top plan view of the embodiment shown in FIG. 1.

FIG. 3 is a cross-sectional view of the embodiment shown in FIG. 1, taken along line 3-3.

FIG. 4 is an exploded perspective view of the embodiment shown in FIG. 1.

FIG. 5 is an exploded elevational view of the embodiment shown in FIG. 1.

FIG. 6A-6B are schematic diagrams showing extension and retraction of the needles.

FIG. 7A-7C are photographs of a micro-needling system and a hand-held rotational unit.

### DETAILED DESCRIPTION OF THE INVENTION

A preferred embodiment of the invention is now described in detail. Referring to the drawings, like numbers indicate like parts throughout the views. Unless otherwise specifically indicated in the disclosure that follows, the drawings are not necessarily drawn to scale. As used in the description herein and throughout the claims, the following terms take the meanings explicitly associated herein, unless the context clearly dictates otherwise: the meaning of “a,” “an,” and “the” includes plural reference, the meaning of “in” includes “in” and “on.”

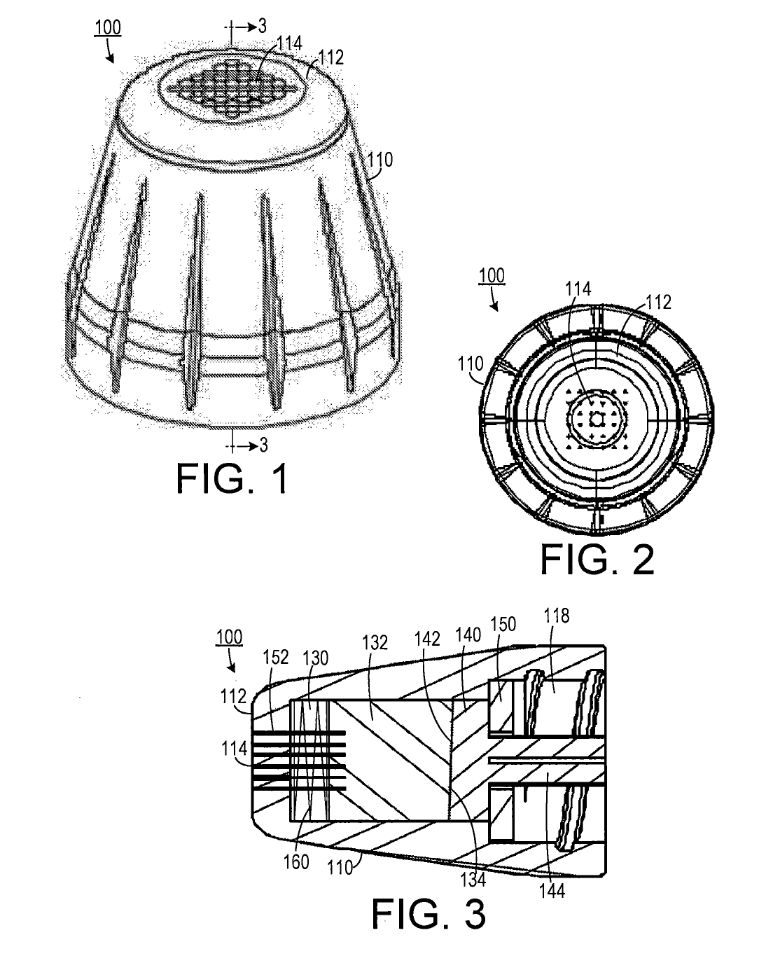

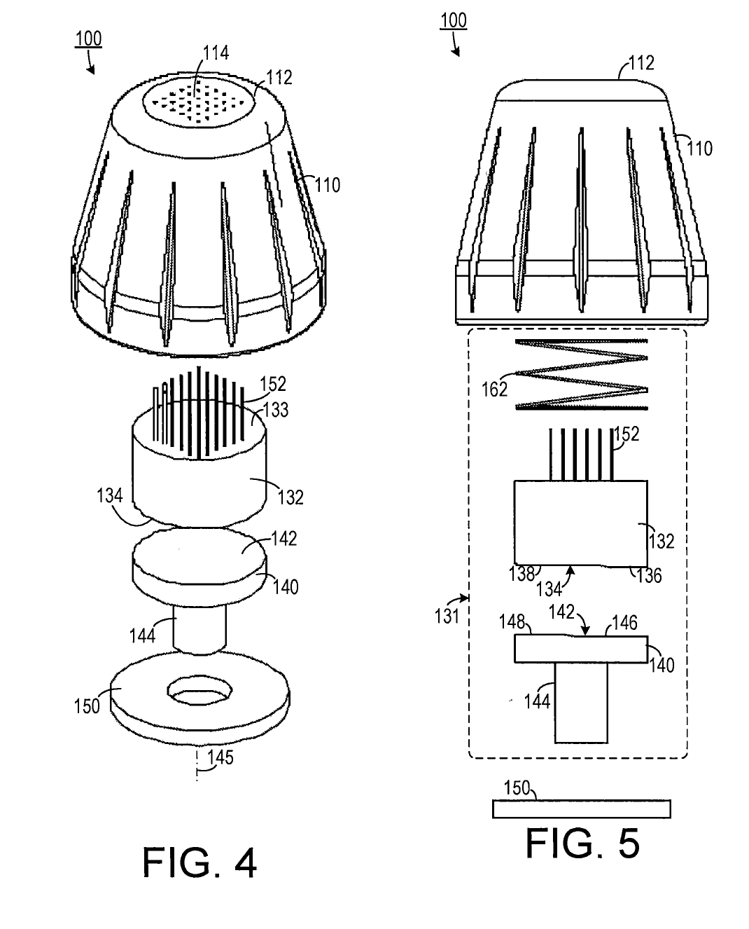

As shown in FIGS. 1-5, one embodiment of a micro-needling attachment 100, which can be attached to a hand-held motorized unit 160—such as a handheld rotational unit of a PMD system (such as a system of the type that can be purchased from PMD Beauty, 12441 South 900 East Ste. 75, Draper, Utah 84020, www.pmdbeauty.com)—includes an outer shell 110 with a top surface 112 that defines a plurality of needle holes 114 passing therethrough. The outer shell 110 defines a concentric cylindrical cavity 130 and the outer shell 110 can include one of many different materials, such as: acrylic, acrylonitrile butadiene styrene, and polyethylene. Typically, the outer shell 110 is injection molded and the needle holes 114 are either formed by a mold or drilled after molding. In alternate embodiments, it can be made from metal, such as stamped or extruded/machined aluminum.

A translation unit 131 that translates the motion from the hand-held motorized unit 160 into reciprocal oscillating linear motion fits into the cavity 130. The translation unit 131 can include, for example, a plug member 132 and a rotatable lower rotary cam 140 that are disposed in the cavity 130. A plurality of needles 152 extend from a flat surface 133 of the plug member 132 and pass through the needle holes 114. A spring 162 maintains the plug member 132 and the needles 152 in a retracted position unless an outward force is applied to them. An axle 144 extends inwardly from the lower rotary cam 140 and passes through a holding retainer 150, which holds the lower rotary cam 140 and the plug 132 within the cavity 130. The rotary cam is configured to rotate about an axis 145 and engage a corresponding rotational member in the hand held rotational unit 160. The plug 132 includes an uneven bottom surface 134 with a base portion 138 and a raised portion 136. Similarly, the lower rotary cam 140 includes an uneven upper cam surface 142 with a base portion 146 and a raised portion 148.

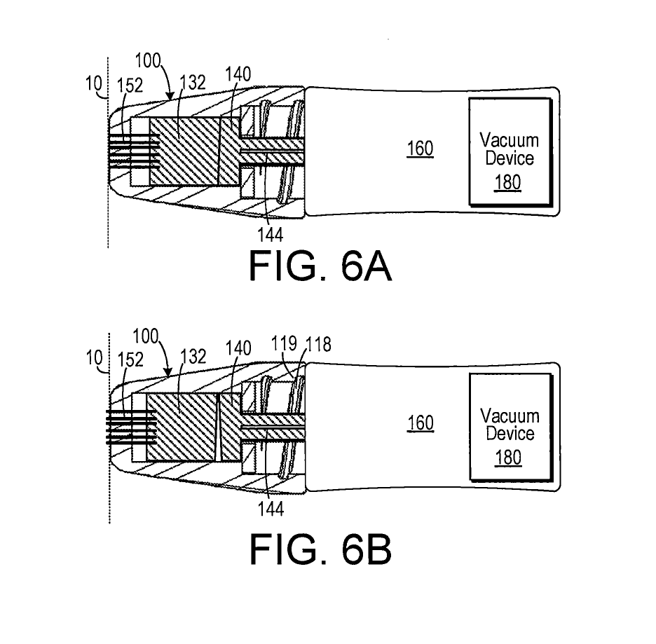

As shown in FIGS. 6A and 6B, one embodiment includes the hand held rotational unit 160 integrated with the micro-needling attachment 100. The cavity 130 can also include a threaded portion 118 to facilitate attachment of the unit 100 to a hand-held rotational unit, which also includes a complementary threaded portion 119. In operation, as the lower rotary cam 140 rotates while driven by the hand held rotational unit 160, raised portion 148 comes into and out of alignment with the raised portion 136 of the plug 132. When in they are in alignment, the plug 132 and, thus, the needles 152 are pushed outwardly into the skin 10. When the raised portion 148 is in alignment with base portion 138, the spring 160 pushes the plug 132 and the needles 152 inwardly, away from the skin 10. Thus, a continuous rotation cycle of the axle 144 results in a continuous reciprocal motion cycle applied to the needles 152. The hand-held rotational unit 160 can also include a suction mechanism/vacuum device 180 that reduces air pressure within the cavity 130. The reduced air pressure applies suction through the holes 114 to the skin 10, thereby keeping the skin in contact with surface 112 and resulting in more consistent extension of the needles 152 into the skin 10.

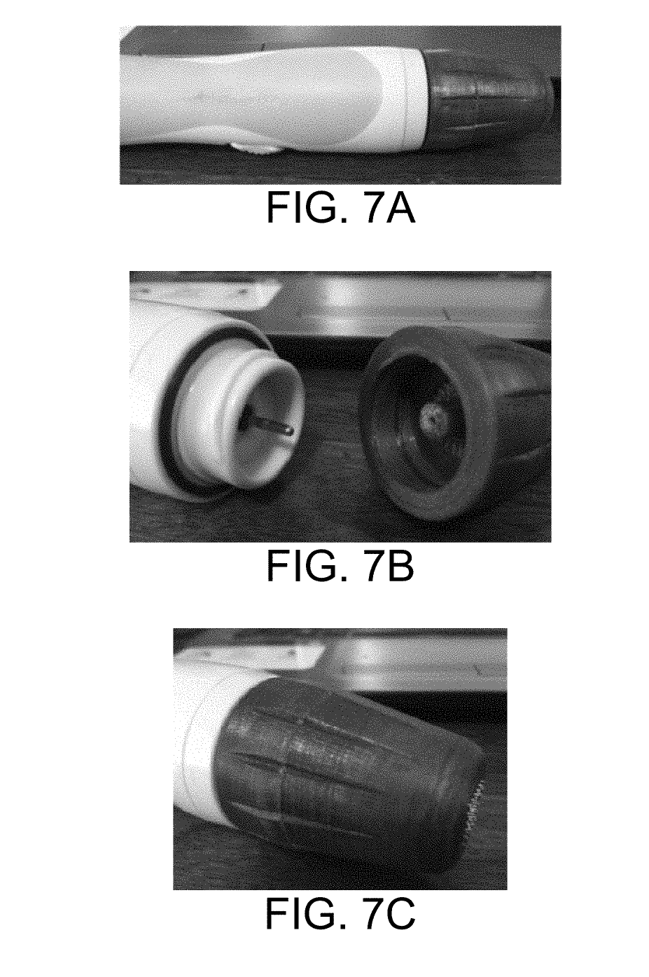

One example of an experimental unit is shown in FIGS. 7A-7C.

The unit offers several advantages, including: it provides a vacuum through the holes where the needles protrude out of the unit, which draws the skin up against the top surface, to maximize the effectiveness of the needles. This application promotes a positive contact with the user's skin and eliminates a bouncing affect by the dermal layer, when the needles are moving in and out at high speed. This insures that the needles are reaching the proper depth when the procedure is done. It is easy to change over from microdermabrasion attachment to the needling attachment for the PMD unit. Micro-needling provides a method to deliver and maximize the benefits of topical creams and lotions. The micro-needling attachment can be made disposable and intended for a one time use only.

In operation, when the motor of the PMD unit rotates, it will cause the lower rotary cam to rotate. The rotary cam has an angled face in which to act as a cam. The Needle assembly also has an angled face on the bottom, that rests against the top face of the lower rotary cam. When activated, this assembly will drive the needle assembly in a vertical motion. The needle assembly, also has flat areas on the side, to keep the needle assemble from rotating when in operation.

The spring in the assembly, acts to retract the needles back into the tip, as the assembly slides across the skin. The upper face of the tip is thick enough to not allow the needles to retract and not leave the engagement of the holes therein. A vacuum is allowed to flow around the inside of the unit while in operation. The lower retainer can be bonded or fastened in place to complete the assembly.

In alternate embodiments, the oscillating reciprocal motion of the needles 152 can be induced by such devices as a solenoid or an electronically-controlled diaphragm.

The above described embodiments, while including the preferred embodiment and the best mode of the invention known to the inventor at the time of filing, are given as illustrative examples only. It will be readily appreciated that many deviations may be made from the specific embodiments disclosed in this specification without departing from the spirit and scope of the invention. Accordingly, the scope of the invention is to be determined by the claims below rather than being limited to the specifically described embodiments above.

What is claimed is:

1. A micro-needling attachment for use with a hand-held motorized unit, comprising:
(a) an outer shell defining a cylindrical cavity therein and having a top surface, the top surface defining a plurality of needle holes passing therethrough, the outer shell couplable to the hand-held motorized unit;
(b) a cylindrical plug member disposed within the cylindrical cavity;
(c) a plurality of needles extending upwardly from the plug member through the needle holes; and
(d) a translation unit that translates motion of a first type from the hand-held motorized unit into oscillating inward and outward motion applied to the plug member so as to cause the plurality of needles to oscillate between an outward position in which the needles extend beyond the outer shell and an inward position.

2. The micro-needling attachment of claim 1, wherein the hand-held rotational unit generates rotational motion and wherein the cylindrical plug member has a flat surface from which the needles extend and an opposite uneven bottom surface that includes a base portion and a raised portion and wherein the translation unit comprises a rotatable lower rotary cam disposed adjacent to the uneven bottom surface of the plug member, an axle depending downwardly therefrom that is engageable with and configured to receive rotational motion from the hand-held motorized unit, the rotary cam having an uneven cam surface that causes the plug member to oscillate between the outward position and the inward position as the rotary cam rotates about an axis.

3. The micro-needling attachment of claim 2, further comprising a spring, disposed between the top surface of the outer shell and the flat surface of the cylindrical plug, that imparts a separating force between the top surface of the outer shell and the flat surface of the cylindrical plug so as to push the plug member into the inward position during a portion the rotary cam rotation cycle.

4. The micro-needling attachment of claim 3, further comprising a holding retainer that holds the rotary cam and the plug within the cavity.

5. The micro-needling attachment of claim 1, wherein hand-held motorized unit includes a threaded end and further comprising a threaded portion disposed in cylindrical cavity of the outer shell that is complementary in shape to the threaded end so as to facilitate attachment thereto.

6. The micro-needling attachment of claim 1, wherein the hand-held motorized unit comprises personal micro dermabrasion unit.

7. The micro-needling attachment of claim 1, wherein the outer shell comprises a material selected from a list of materials consisting of: acrylic, acrylonitrile butadiene styrene, and polyethylene.

8. A micro-needling attachment device for use with a hand-held motorized unit, comprising:
(a) an outer shell defining a cylindrical cavity therein and having a top surface, the top surface defining a plurality of needle holes passing therethrough, the outer shell couplable to the hand-held motorized unit;
(b) a cylindrical plug member disposed within the cylindrical cavity, the cylindrical plug member having a flat surface and an opposite uneven bottom surface that includes a base portion and a raised portion;
(c) a plurality of needles extending upwardly from the flat surface of the plug member through the needle holes;
(d) a rotatable lower rotary cam disposed adjacent to the uneven bottom surface of the plug member, an axle depending downwardly therefrom that is engageable with and configured to receive rotational motion from the hand-held motorized unit, the rotary cam having an uneven cam surface that causes the plug member to oscillate between an outward position in which the needles extend beyond the outer shell and an inward position as the rotary cam rotates about an axis; and
(e) a spring, disposed between the top surface of the outer shell and the flat surface of the cylindrical plug, that imparts a separating force between the top surface of the outer shell and the flat surface of the cylindrical plug so as to push the plug member into the inward position during a portion the rotary cam rotation cycle.

9. The micro-needling attachment device of claim 8, further comprising a holding retainer that holds the rotary cam and the plug within the cavity.

10. The micro-needling attachment device of claim 8, wherein hand-held motorized unit includes a threaded end and further comprising a threaded portion disposed in cylindrical cavity of the outer shell that is complementary in shape to the threaded end so as to facilitate attachment thereto.

11. The micro-needling attachment device of claim 8, wherein the hand-held motorized unit comprises personal micro dermabrasion unit.

12. The micro-needling attachment device of claim 8, wherein the outer shell comprises a material selected from a list of materials consisting of: acrylic, acrylonitrile butadiene styrene, and polyethylene.

13. A micro-needling device, comprising:
(a) a hand-held motorized unit that generates repetitive motion;
(b) an outer shell coupled to the hand-held motorized unit and defining a cylindrical cavity therein and having a top surface, the top surface defining a plurality of needle holes passing therethrough;
(c) a cylindrical plug member disposed within the cylindrical cavity;
(d) a plurality of needles extending upwardly from the plug member through the needle holes;
(e) a translation unit that translates the repetitive motion from the hand-held motorized unit into oscillating inward and outward motion applied to the plug member so as to cause the plurality of needles to oscillate between an outward position in which the needles extend beyond the outer shell and an inward position.

14. The micro-needling device of claim 13, wherein the hand-held motorized unit generates rotational motion and wherein the cylindrical plug member has a flat surface and an opposite uneven bottom surface that includes a base portion and a raised portion and wherein the translation unit comprises a rotatable lower rotary cam disposed adjacent to the uneven bottom surface of the plug member, an axle depending downwardly therefrom that is engageable with and configured to receive rotational motion from the hand-held motorized unit, the rotary cam having an uneven cam surface that causes the plug member to oscillate between the outward position and the inward position as the rotary cam rotates about an axis.

15. The micro-needling device of claim 14, further comprising a spring, disposed between the top surface of the outer shell and the flat surface of the cylindrical plug, that imparts a separating force between the top surface of the outer shell and the flat surface of the cylindrical plug so as to push the plug member into the inward position during a portion the rotary cam rotation cycle.

16. The micro-needling attachment of claim 15, further comprising a holding retainer that holds the rotary cam and the plug within the cavity.

17. The micro-needling device of claim 13, wherein hand-held motorized unit includes a threaded end and further comprising a threaded portion disposed in cylindrical cavity of the outer shell that is complementary in shape to the threaded end so as to facilitate attachment thereto.

18. The micro-needling device of claim 13, wherein the hand-held motorized unit comprises personal micro dermabrasion unit.

19. The micro-needling device of claim 13, wherein the outer shell comprises a material selected from a list of materials consisting of: acrylic, acrylonitrile butadiene styrene, and polyethylene.
